(19) United States
(12) Patent Application Publication
(10) Pub. No.: US 2014/0066773 A1
(43) Pub. Date: Mar. 6, 2014

(54) ULTRASONIC DIAGNOSIS APPARATUS AND IMAGE PROCESSING APPARATUS

(71) Applicants: Toshiba Medical Systems Corporation, Otawara-shi (JP); Kabushiki Kaisha Toshiba, Minato-ku (JP)

(72) Inventor: Masao TAKIMOTO, Otawara-shi (JP)

(73) Assignees: Toshiba Medical Systems Corporation, Otawara-shi (JP); Kabushiki Kaisha Toshiba, Minato-ku (JP)

(21) Appl. No.: 14/076,804

(22) Filed: Nov. 11, 2013

Related U.S. Application Data
(63) Continuation of application No. PCT/JP2013/067086, filed on Jun. 21, 2013.

(30) Foreign Application Priority Data
Jul. 18, 2012 (JP) 2012-159203

(51) Int. Cl. A61B 8/08; A61B 8/00

(52) U.S. Cl. CPC A61B 8/5207; A61B 8/488; A61B 8/54; A61B 8/461; A61B 8/467; A61B 8/5223
USPC 600/453

(57) ABSTRACT

An ultrasound diagnostic apparatus includes a transmitting and receiving unit, an extracting unit, a generating unit, a brightness information controlling unit and a display unit. The transmitting and receiving unit transmits an ultrasonic wave to and receives an ultrasonic wave from a diagnosis part including a moving body in an object. The extracting unit extracts a Doppler signal based on a reception signal obtained by the transmitting and receiving unit. The generating unit executes a frequency analysis on the basis of the Doppler signal and generates a Doppler spectrum. The brightness information controlling unit controls brightness information on the Doppler spectrum on the basis of a brightness correction value appropriate for a frequency subjected to the frequency analysis. The display unit displays, on a display device, the Doppler spectrum on which the brightness information is controlled by the brightness information controlling unit.

### CROSS-REFERENCE TO RELATED APPLICATION

This application is a Continuation Application of No. PCT/JP2013/67086, filed on Jun. 21, 2013, and the PCT application is based upon and claims the benefit of priority from Japanese Patent Application No. 2012-159203, filed on Jul. 18, 2012, the entire contents of which are incorporated herein by reference.

### FIELD

An embodiment that is an aspect of the present invention relates to an ultrasonic diagnosis apparatus and an image processing apparatus capable of providing a waveform of a Doppler spectrum, which is easy for an operator to measure.

### BACKGROUND

A known prior-art ultrasonic diagnosis apparatus uses the ultrasonic pulse echo method and the ultrasonic Doppler method in combination, obtains a tomographic image of and blood flow information on a diagnosis part by ultrasonic scan using an ultrasonic probe and displays the blood flow information in real time. The principle of the ultrasonic Doppler method consists in that the reception frequency slightly deviates from the transmission frequency because of the Doppler effect of the ultrasonic wave transmitted to and received from the diagnosis part including a flow, such as a blood flow in the body of an object, and the deviated frequency (Doppler frequency) is in proportion to the blood flow velocity. The ultrasonic diagnosis apparatus using the ultrasonic Doppler method can execute a frequency analysis of the Doppler frequency based on the principle and obtain the blood information from the result of the analysis.

The ultrasonic diagnosis apparatus using the ultrasonic Doppler method is used for examination of a patient having a cardiac valve disease. An operator, such as a doctor, can determine the severity of the cardiac valve disease of the patient by using the ultrasonic diagnosis apparatus using the ultrasonic Doppler method. In that case, the examination is executed in the following procedure.

The operator observes a motion of the whole of a heart or a motion of a valve in a B-mode or M-mode and then observes the status of a regurgitation caused by a valve insufficiency in a color mode. Next, in order to see the degree of the regurgitation, the operator makes the mode of the ultrasonic diagnosis apparatus transition from the color mode to a pulsed wave Doppler (PWD) mode or a steerable continuous wave Doppler (SCWD) mode, sets a range gate (RG) position or a focus position (mark position) on the regurgitating blood flow or a blood inflow or outflow, and observes the displayed waveform of the Doppler spectrum. In this process, the RG position or focus position are typically set in the vicinity of a valve.

Furthermore, using a measurement function of the ultrasonic diagnosis apparatus, the operator measures a maximum flow velocity value or a time interval of the blood flow waveform or determines the volume of the regurgitating blood by tracing an envelope of the blood flow waveform, thereby determining the severity of the valve disease of the object. In examination of a patient having a cardiac valve disease, to know the maximum flow velocity value or the volume of the regurgitating blood is very important for the operator to determine the severity of the patient.

However, even if the RG position or focus position is set close to a valve orifice from which a jet of blood regurgitates, for various reasons, the resulting Doppler waveform (a Doppler spectrum waveform) is not always easy to see the regurgitation at frequencies close to 0 Hz nor easy to see the maximum flow velocity value of the regurgitation, and it has been impossible to provide a Doppler spectrum waveform that is easy to trace an envelope of the regurgitation.

A first of the reasons described above is a reason concerning the living body of the object: the blood regurgitation is not always ejected in a fixed direction. A second reason is a reason concerning the performance of the ultrasonic diagnosis apparatus: the sensitivity or the S/N ratio of a signal (in particular, a high frequency regurgitation jet signal) of the ultrasonic diagnosis apparatus. A third reason concerns the skill of the operator to scan the object with an ultrasonic wave using the ultrasonic diagnosis apparatus.

A fourth reason is that the Doppler signal contains a clutter signal, which is an unwanted permanent echo signal from a cardiac wall or the like. Of course, the unwanted low frequency clutter signal can be removed from the Doppler signal from blood cells by using a wall filter. The wall filter eliminates the unwanted low frequency clutter signal by changing the cut-off frequency. In that case, however, depending on the characteristics of the wall filter, the required Doppler signal can also be eliminated along with signal information on a blood flow in the vicinity of a valve orifice, and it can be difficult to execute the time-interval measurement or the like in the ultrasonic Doppler method. In addition, it is complicated to change the filter order of the wall filter or to change the shift frequency fc of the clutter, and it is difficult to change them for each examination.

A method of controlling a Doppler gain without using the wall filter can also be used. However, the method involves an operation of raising or lowering the brightness value over the entire frequency band, and therefore, the required blood flow signal can be less legible as in the case where the wall filter is used.

As described above, for various reasons, it has been difficult to provide a waveform of a Doppler spectrum that is easy for an operator to measure.

### BRIEF DESCRIPTION OF THE DRAWINGS

In accompanying drawings,

FIG. 1 is a block diagram showing a configuration of the whole of the ultrasonic diagnosis apparatus according to the present embodiment;

FIG. 2 is a block diagram showing a detailed configuration of a transmitting and receiving unit and a data generating unit in the ultrasonic diagnosis apparatus according to the present embodiment;

FIG. 3 is a diagram showing a Doppler spectrum waveform according to prior art;

FIG. 4 is a flowchart showing a brightness information controlling processing executed by the ultrasonic diagnosis apparatus according to the present embodiment;

FIG. 5 is a diagram showing a Doppler spectrum waveform according to prior art for which only a uniform brightness correction (adjustment) of a scale value, a baseline value, a gain value and the like has been executed;

FIG. 6 is a diagram showing a state where no brightness correction is executed, which corresponds to a state according to prior art;

FIG. 7 is a diagram showing a first example of a method of controlling the brightness information on the waveform of the Doppler spectrum;

FIG. 8 is a diagram showing a Doppler spectrum waveform in a case where a regurgitation jet signal is captured in the Doppler mode, tip end parts of the regurgitation jet signal are truncated, and the maximum flow velocity value is hard to see;

FIG. 9 is a diagram showing a second example of a method of controlling the brightness information on the waveform of the Doppler spectrum;

FIG. 10 is a diagram for illustrating a method of operating brightness correction values in the case of the controlling method shown in FIG. 7;

FIG. 11 is a diagram showing an example of a user interface for operating brightness correction values in a frequency axis direction;

FIG. 12 is a diagram showing a waveform of a Doppler spectrum having been subjected to the brightness information control in the frequency axis direction based on the brightness correction values shown in FIG. 10;

FIG. 13 is a diagram showing a case where the brightness of signals in low frequency component regions close to 0 Hz is decreased according to a conventional method that involves raising the cut-off setting of the wall filter;

FIG. 14 is a diagram for illustrating a method of operating brightness correction values in the case of the controlling method shown in FIG. 9;

FIG. 15 is a diagram showing a waveform of a Doppler spectrum having been subjected to the brightness information control in the frequency axis direction based on the brightness correction values shown in FIG. 14;

FIG. 16 is a diagram for illustrating a method of operating brightness correction values in the case of the controlling methods shown in FIGS. 7 and 9;

FIG. 17 is a diagram showing a waveform of a Doppler spectrum having been subjected to the brightness information control in the frequency axis direction based on the brightness correction values shown in FIG. 16;

FIG. 18 is a diagram showing a specific example of a gamma curve control that involves operating a gamma curve value;

FIG. 19 is a diagram showing a specific example of a gamma curve control that involves operating a gamma curve value;

FIG. 20 is a diagram for illustrating a specific example of the wall filter control;

FIG. 21 is a diagram showing a waveform of a Doppler spectrum having been subjected to the brightness information control in the frequency axis direction and a time axis direction;

FIG. 22 is a diagram showing an example of the user interface for operating the brightness correction values in the frequency axis direction and the time axis direction; and

FIG. 23 is a block diagram showing a configuration of the whole of an image processing apparatus according to the present embodiment.

### DETAILED DESCRIPTION

An ultrasonic diagnosis apparatus and an image processing apparatus according to the present embodiment will be described with reference to the accompanying drawings.

To solve the above-described problems, the present embodiments provide the ultrasonic diagnosis apparatus, including: a transmitting and receiving unit configured to transmit an ultrasonic wave to and receive an ultrasonic wave from a diagnosis part including a moving body in an object; an extracting unit configured to extract a Doppler signal based on a reception signal obtained by the transmitting and receiving unit; a generating unit configured to execute a frequency analysis on the basis of the Doppler signal and generate a Doppler spectrum; a brightness information controlling unit configured to control brightness information on the Doppler spectrum on the basis of a brightness correction value appropriate for a frequency subjected to the frequency analysis; and a display unit configured to display, on a display device, the Doppler spectrum on which the brightness information is controlled by the brightness information controlling unit.

To solve the above-described problems, the present embodiments provide the image processing apparatus, including: a brightness information controlling unit configured to control brightness information on a Doppler spectrum on the basis of a brightness correction value appropriate for a frequency subjected to a frequency analysis based on a Doppler signal; and a display unit configured to display, on a display device, the Doppler spectrum on which the brightness information is controlled by the brightness information controlling unit.

The ultrasound diagnostic apparatus and the image processing apparatus according to the present embodiment can more quickly execute measurement or other processings and improve the throughput of examination itself.

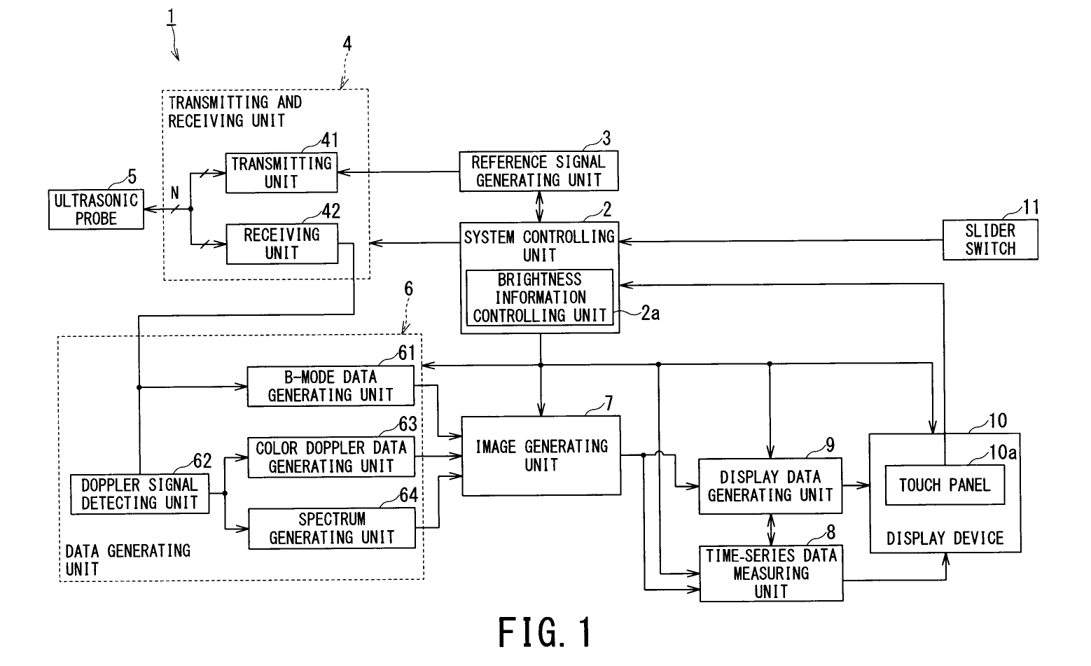

FIG. 1 is a block diagram showing a configuration of the whole of the ultrasonic diagnosis apparatus according to the present embodiment. The ultrasonic diagnosis apparatus according to the present embodiment can operate in various modes, such as a B-mode, in which the apparatus displays an ultrasonic tomographic image (B-mode tomographic image), an M-mode, in which the apparatus displays a temporal variation of a position of a reflection source in a direction of an ultrasonic beam as a motion curve, a Doppler mode (a pulsed wave Doppler (PWD) mode and a continuous wave Doppler (CWD) mode), in which the apparatus displays blood flow information, and a color flow mapping (CFM) or color Doppler mode, in which the apparatus two-dimensionally displays blood flow information.

FIG. 1 shows an ultrasonic diagnosis apparatus 1 according to the present embodiment. The ultrasonic diagnosis apparatus 1 includes a system controlling unit 2, a reference signal generating unit 3, a transmitting and receiving unit 4, an ultrasonic probe 5, a data generating unit 6, an image generating unit 7, a time-series data measuring unit 8, a display data generating unit 9 and a display device 10.

The system controlling unit 2 has a central processing unit (CPU) and a memory. The system controlling unit 2 centrally controls each unit in the ultrasonic diagnosis apparatus 1. In particular, the system controlling unit 2 has a brightness information controlling unit 2a as a characteristic component of the ultrasonic diagnosis apparatus 1 according to the present embodiment.

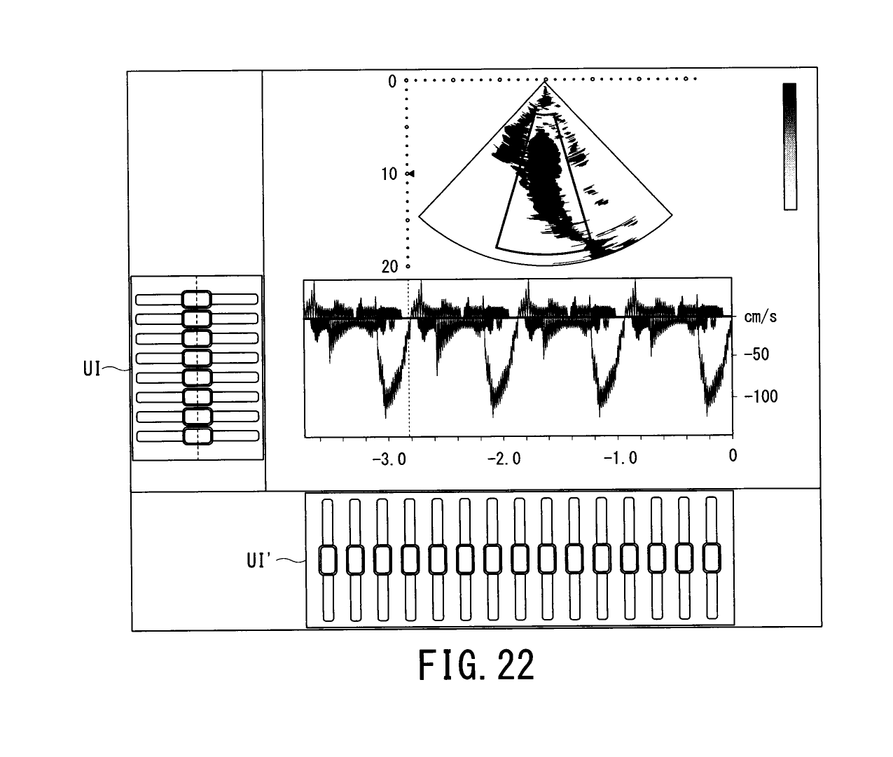

The brightness information controlling unit 2a controls the display data generating unit 9 described later, determines a brightness correction value appropriate for a frequency (a frequency axis direction) subjected to a frequency analysis in accordance with an instruction from a touch panel 10a (a slider switch 11), and controls brightness information on a Doppler spectrum (a Doppler spectrum image) based on the brightness correction value. The brightness information controlling unit 2a desirably determines the brightness correction value for each of a plurality of frequency domains resulting from division of frequency band. Furthermore, as described later with reference to FIGS. 21 and 22, the brightness information controlling unit 2a can determine a second brightness correction value appropriate for time (in a time axis direction) in addition to the brightness correction value appropriate for the frequency, and control the brightness information on the Doppler spectrum based on the second brightness correction value. The brightness information controlling unit 2a desirably determines the second brightness correction value for each of a plurality of time domains resulting from division of a time period.

The reference signal generating unit 3 generates a continuous wave or rectangular wave at a frequency approximately equal to a central frequency of an ultrasonic pulse, for example, for the transmitting and receiving unit 4 and the data generating unit 6 in accordance with a control signal from the system controlling unit 2.

The transmitting and receiving unit 4 transmits a signal to and receives a signal from the ultrasonic probe 5. The transmitting and receiving unit 4 includes a transmitting unit 41 that generates a drive signal that makes the ultrasonic probe 5 emit a transmission ultrasonic wave and a receiving unit 42 that executes a phasing addition of reception signals from the ultrasonic probe 5.

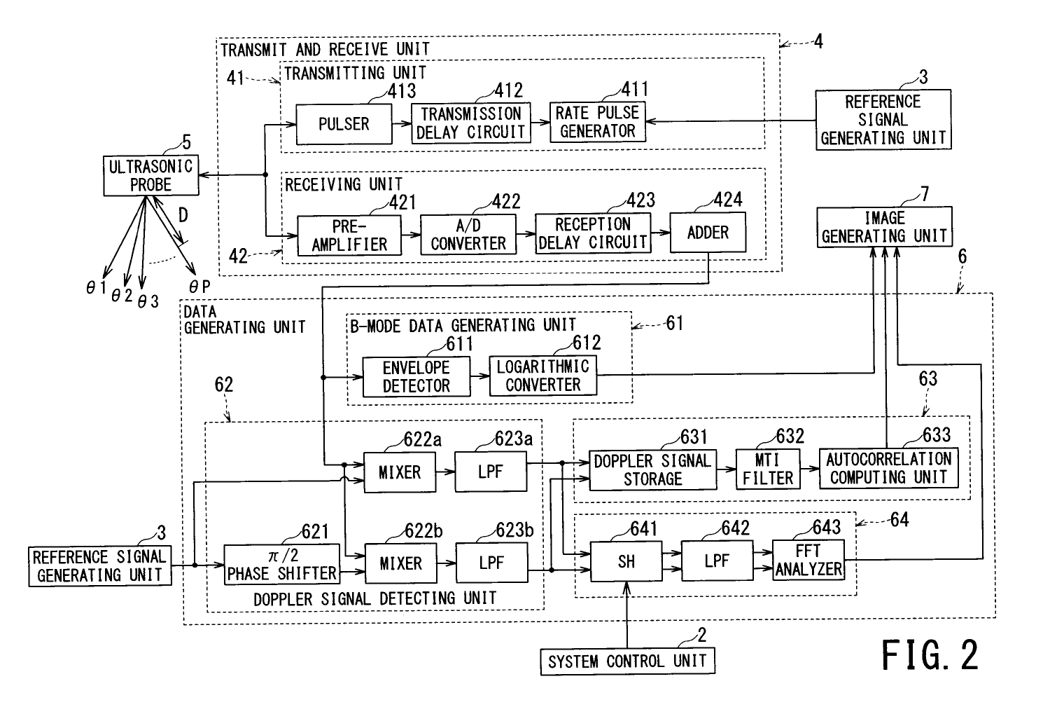

FIG. 2 is a block diagram showing a detailed configuration of the transmitting and receiving unit 4 and the data generating unit 6 in the ultrasonic diagnosis apparatus 1 according to the present embodiment.

As shown in FIG. 2, the transmitting and receiving unit 4 includes a rate pulse generator 411, a transmission delay circuit 412, and a pulser 413. The rate pulse generator 411 generates a rate pulse that determines a repetition period of the transmission ultrasonic wave by dividing the frequency of the continuous wave or rectangular wave supplied from the reference signal generating unit 3, and supplies the rate pulse to the transmission delay circuit 412.

The transmission delay circuit 412 is formed by the same number of (N channels of) independent delay circuits as the ultrasonic transducers used for transmission. The transmission delay circuit 412 imparts, to the rate pulse, a delay time for making the ultrasonic wave converge at a predetermined depth in order to provide a narrow beam width in transmission and a delay time for emitting the transmission ultrasonic wave in a predetermined direction, and supplies the rate pulse to the pulser 413.

The pulser 413 has N channels of independent driving circuits, and generates a drive pulse for driving the ultrasonic transducers in the ultrasonic probe 5 based on the rate pulse.

Referring back to FIG. 1, the ultrasonic probe 5 transmits an ultrasonic wave to and receives an ultrasonic wave from an object. The ultrasonic probe 5 executes transmission and reception of an ultrasonic wave with a front surface thereof in contact with a surface of the object, and has a plurality of (N) minute ultrasonic transducers one-dimensionally arranged on a tip end part thereof. The ultrasonic transducers are electroacoustic conversion elements and have a capability of converting an electric pulse into an ultrasonic pulse (a transmission ultrasonic wave) in transmission and converting a reflection ultrasonic wave (a reception ultrasonic wave) into an electric signal (a reception signal) in reception.

The ultrasonic probe 5 is small and light and is connected to the transmitting unit 41 and the receiving unit 42 in the transmitting and receiving unit 4 by cables. The ultrasonic probe 5 may be of the sector scanning type, the linear scanning type, the convex scanning type or other types, which can be appropriately chosen depending on the diagnosis part. In the following, a case where the ultrasonic probe 5 is an ultrasonic probe of the sector scanning type used for cardiac function measurement will be described. However, the present invention is not limited to the type, and the linear scanning type or the convex scanning type can also be used.

As shown in FIG. 2, the receiving unit 42 includes a preamplifier 421, an analog to digital (A/D) converter 422, a reception delay circuit 423, and an adder 424. The preamplifier 421 is formed by N channels and amplifies weak reception electric signals produced by conversion by the ultrasonic transducers to ensure an adequate S/N ratio. The reception signals of the N channels amplified to a predetermined magnitude by the preamplifier 421 are converted into digital signals by the A/D converter 422, and the resulting digital signals are passed to the reception delay circuit 423.

The reception delay circuit 423 imparts, to each of the reception signals of the N channels output from the A/D converter 422, a focusing delay time for focusing a reflection ultrasonic wave from a predetermined depth and a deflecting delay time for setting a reception directivity in a predetermined direction.

The adder 424 executes a phasing addition of reception signals from the reception delay circuit 423 (an addition of the reception signals from the predetermined direction in phase with each other).

Referring back to FIG. 1, the data generating unit 6 generates B-mode data, color Doppler data and a Doppler spectrum based on the reception signals obtained from the transmitting and receiving unit 4.

As shown in FIG. 2, the data generating unit 6 includes a B-mode data generating unit 61, a Doppler signal detecting unit 62, a color Doppler data generating unit 63, and a spectrum generating unit 64. The B-mode data generating unit 61 generates B-mode data for the reception signal output from the adder 424 in the receiving unit 42. The B-mode data generating unit 61 includes an envelope detector 611 and a logarithmic converter 612. The envelope detector 611 executes an envelope detection of the reception signal produced by the phasing addition supplied from the adder 424 in the receiving unit 42, and the logarithmic converter 612 executes a logarithmic conversion of the amplitude of the resulting envelope detection signal.

The Doppler signal detecting unit 62 detects (extracts) a Doppler signal by executing an orthogonal detection of the reception signal. The Doppler signal detecting unit 62 includes a π/2 phase shifter 621, mixers 622a and 622b, and low pass filters (LPFs) 623a and 623b. The Doppler signal detecting unit 62 detects a Doppler signal by executing an orthogonal phase detection of the reception signal supplied from the adder 424 in the receiving unit 42.

The color Doppler data generating unit 63 generates color Doppler data based on the detected Doppler signal. The color Doppler data generating unit 63 includes a Doppler signal storage 631, a moving target indicator (MTI) filter 632, and an autocorrelation calculator 633. The Doppler signal from the Doppler signal detecting unit 62 is temporarily stored in the Doppler signal storage 631.

The MTI filter 632, which is a high pass digital filter, reads the Doppler signal stored in the Doppler signal storage 631 and removes a Doppler component (a clutter component) caused by a respiration-induced or pulsation-induced movement of an organ from the Doppler signal.

The autocorrelation calculator 633 calculates an autocorrelation value for the blood information solely extracted from the Doppler signal by the MTI filter 632 and calculates an average flow velocity value, a variance value or the like of the blood flow based on the autocorrelation value.

The spectrum generating unit 64 executes an FFT analysis of the Doppler signal obtained by the Doppler signal detecting unit 62 and generates a frequency spectrum (a Doppler spectrum) of the Doppler signal. The spectrum generating unit 64 includes a sample holding (SH) circuit 641, a low pass filter (LPF) 642, and a fast Fourier transform (FFT) analyzer 643. Note that the SH 641 and the LPF 642 are both formed by two channels, to which complex components, a real component (an I component) and an imaginary component (a Q component), of the Doppler signal output from the Doppler signal detecting unit 62 are supplied, respectively.

To the SH 641, the Doppler signals output from the LPFs 623a and 623b in the Doppler signal detecting unit 62 and a sampling pulse (a range gate pulse) generated by the system controlling unit 2 frequency-dividing the reference signal from the reference signal generating unit 3 are supplied. The SH 641 holds samples of the Doppler signal from a desired depth D in accordance with the sampling pulse. Note that the sampling pulse occurs a delay time Ts after the rate pulse that determines the timing of emission of the transmission ultrasonic wave, the delay time Ts can be arbitrarily set.

The LPF 642 removes a stepwise noise component superposed on the Doppler signal from the depth D output from the SH 641.

The FFT analyzer 643 generates a Doppler spectrum based on the supplied smoothed Doppler signal. The FFT analyzer 643 has a calculating circuit and a storage circuit, although not shown. The Doppler signal output from the LPF 642 is temporarily stored in the storage circuit. The calculating circuit generates a Doppler spectrum by executing an FFT analysis of a series of Doppler signals stored in the storage circuit for a predetermined period. Note that the spectrum generating unit 64 may include a wall filter that removes a clutter signal of a low frequency component.

In the case shown in FIG. 2, a pulsed wave Doppler (PWD) mode in which a part of the continuous wave generated by the reference signal generating unit 3 is extracted and used as the transmission wave has been primarily described. However, a continuous wave Doppler (CWD) mode in which the continuous wave is used as the transmission wave is also possible. In the case of the CWD mode, the Doppler signal detecting unit 62 executes the same processing, and the FFT analyzer 64 in the spectrum generating unit 64 then generates a Doppler spectrum by executing the FFT analysis of the Doppler signals output from the LPFs 623a and 623b in the Doppler signal detecting unit 62.

Referring back to FIG. 1, the image generating unit 7 generates a B-mode image and a color Doppler image, which are ultrasonic images, as data by storing the B-mode data and the color Doppler data obtained by the data generating unit 6 associated with each other in a scan direction and generates a Doppler spectrum image and an M-mode image, which are ultrasonic images, as data by storing the Doppler spectrum and the B-mode data obtained in a predetermined scan direction in a time-series manner.

For example, the image generating unit 7 generates the B-mode image and the color Doppler image by sequentially storing the B-mode data and the color Doppler data for each of scan directions θ1 to θP generated by the data generating unit 6 based on the reception signals obtained by transmitting and receiving ultrasonic waves in the scan directions θ1 to θP. In addition, the image generating unit 7 generates the M-mode image by storing in the time-series manner the B-mode data obtained by a plurality of transmissions and receptions of ultrasonic waves in a desired scan direction θp (p=1, 2, . . . , P), and generates the Doppler spectrum image by storing in the time-series manner the Doppler spectra based on the reception signals from a distance D in the scan direction θp obtained by the same receptions of ultrasonic waves as described above. That is, a plurality of B-mode images and a plurality of color Doppler images are stored in an image data storage region of the image generating unit 7, and the M-mode image and the Doppler spectrum image are stored in a time-series data storage region of the image generating unit 7.

The time-series data measuring unit 8 reads time-series data stored in the image generating unit 7 in a predetermined time period and measures a diagnosis parameter, such as a velocity trace, based on the time-series data. Furthermore, the time-series data measuring unit 8 executes various kinds of measurements concerning the Doppler spectrum (such as a time-interval measurement, a measurement of the maximum flow velocity value, a measurement of the blood volume) using the waveform of the Doppler spectrum having been subjected to the brightness control in the frequency axis direction under the control of the system controlling unit 2.

The display data generating unit 9 generates display data by combining an ultrasonic image generated by the image generating unit 7 and a measurement value of a diagnosis parameter measured by the time-series data measuring unit 8 in a predetermined display format.

The display device 10 displays the display data generated by the display data generating unit 9. The display device 10 includes a converting circuit and a display unit (a display) (both not shown) and the touch panel 10a. The converting circuit generates a video signal for display on the display by executing a D/A conversion and a television format conversion of the above-described display data generated by the display data generating unit 9. The touch panel 10a includes a plurality of touch sensors (not shown) and is provided on a display screen of the display. An operator, such as a doctor, can input various kinds of commands to the ultrasonic diagnosis apparatus 1 by operating the touch panel 10a.

Even if the RG position or focus position is set close to a valve orifice from which a jet of blood regurgitates, for various reasons, the resulting Doppler waveform (a Doppler spectrum waveform) is not always easy to see the regurgitation at frequencies close to 0 Hz nor easy to see the maximum flow velocity value of the regurgitation, and it has been impossible to provide a Doppler spectrum waveform that is easy to trace an envelope of the regurgitation.

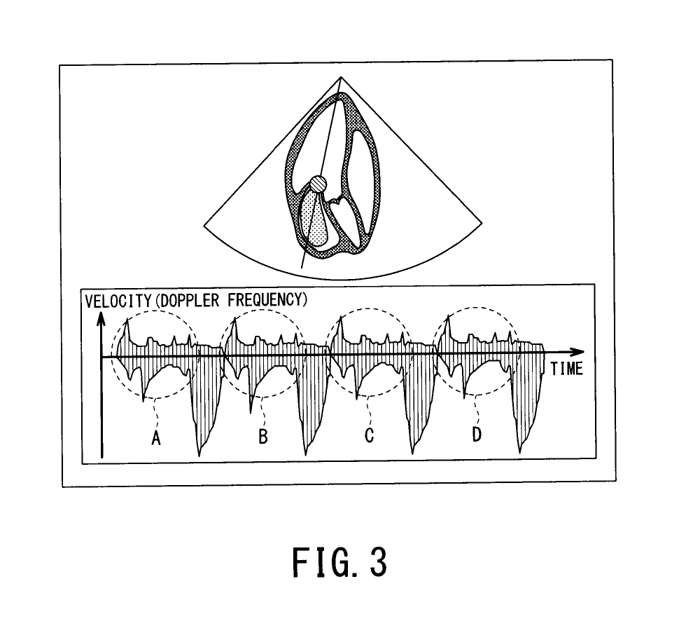

FIG. 3 is a diagram showing a Doppler spectrum waveform according to prior art.

In regions A to D in FIG. 3, the Doppler spectrum waveform is not easy for an operator to execute the measurements, and it disrupts the measurements such as the time-interval measurement, the measurement of the maximum flow velocity value, or the measurement of the volume of the regurgitation blood.

Among other reasons, the dependence of the sensitivity or signal (in particular, a high frequency regurgitation jet signal) of the ultrasonic diagnosis apparatus on the S/N ratio and the inclusion of a clutter signal, which is an unwanted permanent echo from a cardiac wall or the like, in the Doppler signal pose problems. In view of this, a processing for providing a Doppler spectrum waveform that is easy for an operator to measure under the control of the brightness information controlling unit 2a described above will be described below.

Note that the ultrasonic diagnosis apparatus 1 may be provided with the slider switch 11 for brightness information control as an alternative to or in addition to the touch panel 10a. The slider switch 11 is operated by the operator and receives an input command for controlling the Doppler spectrum brightness information. The slider switch 11 supplies information on the received command input by the operator to the brightness information controlling unit 2a in the system controlling unit 2 as required.

Next, a brightness information controlling processing executed by the ultrasonic diagnosis apparatus 1 according to the present embodiment will be described.

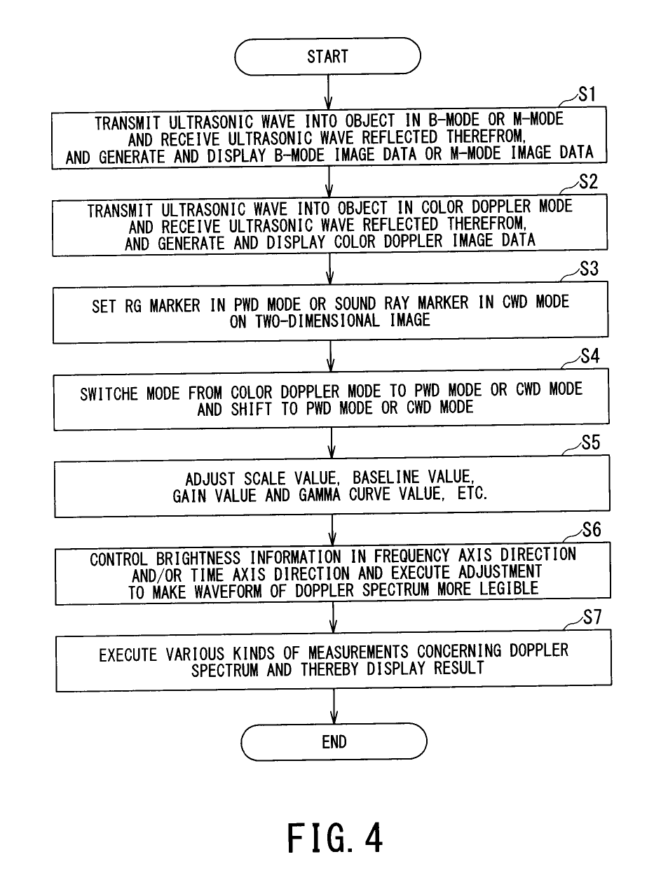

FIG. 4 is a flowchart showing a brightness information controlling processing executed by the ultrasonic diagnosis apparatus 1 according to the present embodiment.

In step S1, the ultrasonic diagnosis apparatus 1 transmits an ultrasonic wave into an object (a diagnosis part including a moving body in the object) in the B-mode or M-mode and receives the ultrasonic wave reflected therefrom and generates B-mode image data or M-mode image data, and the display device 10 of the ultrasonic diagnosis apparatus 1 displays an image based on the B-mode image data or an image based on the M-mode image data. The processing of generating the B-mode image data or the M-mode image data is as described above with reference to FIG. 2 and will not be further described. The operator then observes a motion of the whole of the heart or a motion of a valve in the B-mode or M-mode.

In step S2, the ultrasonic diagnosis apparatus 1 transmits an ultrasonic wave into the object in the color Doppler mode, receives the ultrasonic wave reflected therefrom and generates color Doppler image data, and the display device 10 displays an image based on the color Doppler image data. The processing of generating the color Doppler image data is as described above with reference to FIG. 2 and will not be further described. The operator then observes the status of a regurgitation caused by a valve insufficiency in the color mode.

The operator then inputs settings of an RG marker in the PWD mode or a sound ray marker in the CWD mode on the two-dimensional image, such as the B-mode image, the M-mode image or the color Doppler image, on the touch panel 10a. In step S3, the system controlling unit 2 of the ultrasonic diagnosis apparatus 1 receives the input settings of the RG marker in the PWD mode or the sound ray marker in the CWD mode via the touch panel 10a and sets the RG marker in the PWD mode or the sound ray marker in the CWD mode.

In step S4, the system controlling unit 2 of the ultrasonic diagnosis apparatus 1 switches the mode of the ultrasonic diagnosis apparatus 1 from the color Doppler mode to the PWD mode or CWD mode, thereby shifts to the PWD mode or CWD mode. In the PWD mode, the system controlling unit 2 of the ultrasonic diagnosis apparatus 1 controls the transmitting and receiving unit 4, the data generating unit 6 and the like to receive a signal from inside the set RG marker, generate (extract) a Doppler signal (a Doppler signal induced by the moving body in the object), execute the FFT analysis of the generated Doppler signal and generate a Doppler spectrum. Alternatively, in the CWD mode, the system controlling unit 2 of the ultrasonic diagnosis apparatus 1 controls the transmitting and receiving unit 4, the data generating unit 6 and the like to receive a signal from the set sound ray marker, generate a Doppler signal, execute the FFT analysis of the generated Doppler signal and generate a Doppler spectrum.

In step S5, in the processing of generating the Doppler spectrum, the system controlling unit 2 controls the image generating unit 7, the display data generating unit 9 and the like to uniformly adjust a scale value, a baseline value, a gain value, a gamma curve value, a wall filter value and the like so that the waveform of the generated Doppler spectrum is easy for the operator to measure. Note that the waveform of the Doppler spectrum that is easy for the operator to measure means a waveform for which the signals from blood cells in the Doppler signals can be easily distinguished from the other unwanted signals (such as clutter signals) and the maximum flow velocity value, the blood volume or the like can be easily determined.

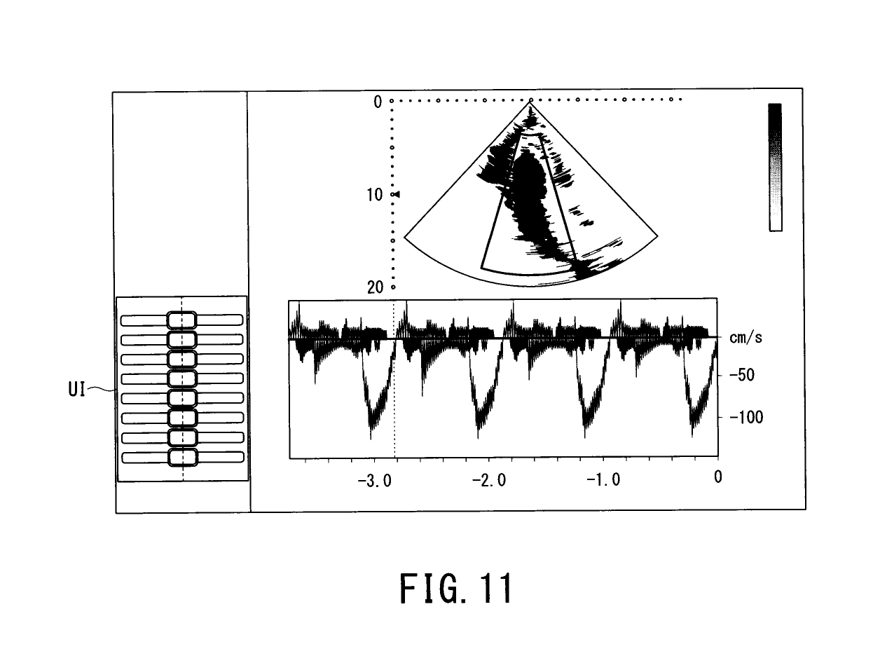

It is then determined whether the waveform of the Doppler spectrum for which the values described above have been uniformly adjusted is easy for the operator to measure or not. If it is determined that the waveform is not easy for the operator to measure, the operator performs a sliding operation to set a slider bar UI (shown in FIG. 11) displayed on the display to a press position. If the touch panel 10a detects a sliding operation from the press position, the brightness information controlling unit 2a of the system controlling unit 2 executes an adjustment to make the waveform of the Doppler spectrum more legible.

In step S6, the brightness information controlling unit 2a of the system controlling unit 2 controls the display data generating unit 9 to control the brightness information in the frequency axis direction with respect to the Doppler spectrum image generated by the image generating unit 7 in accordance with a command input by the operator via the touch panel 10a.

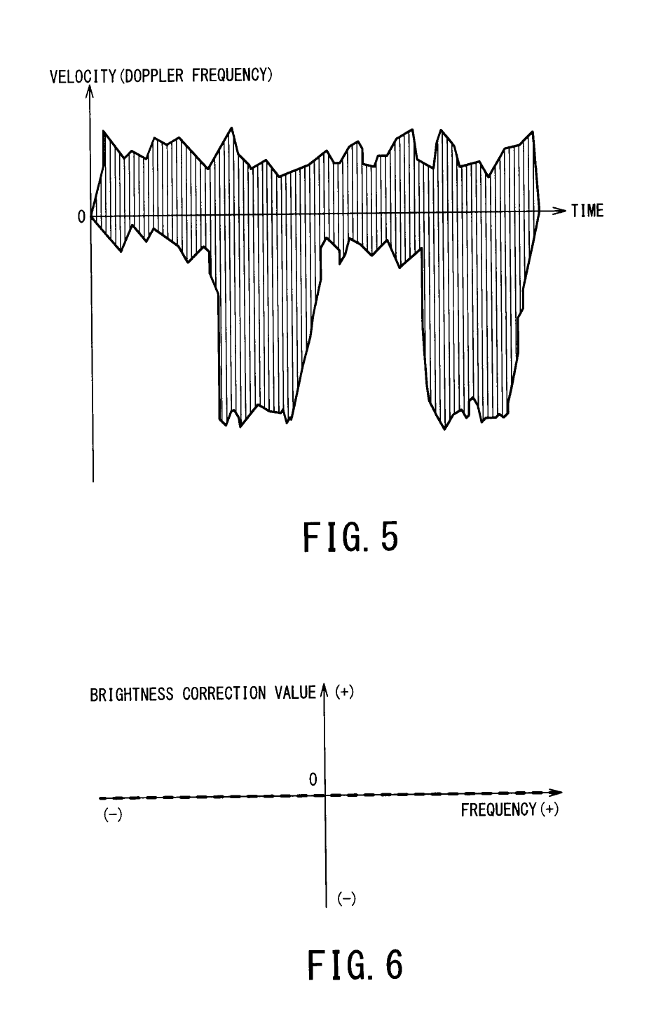

FIG. 5 is a diagram showing a Doppler spectrum waveform according to prior art for which only a uniform brightness correction (adjustment) of the scale value, the baseline value, the gain value and the like has been executed. Note that, since the blood flow velocity is in proportion to the Doppler frequency, the vertical axis indicates the Doppler frequency as well as the blood flow velocity. FIG. 6 is a diagram showing a state where no brightness correction is executed, which corresponds to a state according to prior art.

For the ultrasonic diagnosis apparatus 1 according to the present embodiment, in the brightness control of the waveform of the Doppler spectrum using the brightness correction value by the brightness information controlling function of the brightness information controlling unit 2a, a coordinate system whose horizontal axis is the frequency axis and whose vertical axis indicates the brightness correction value is considered. FIG. 6 shows a case where the brightness correction by the ultrasonic diagnosis apparatus 1 according to the present embodiment is not executed but only a uniform correction of the scale value, the baseline value, the gain value and the like is executed, which corresponds to a state according to prior art.

Next, a method of controlling the brightness information in a case where a blood flow signal in a low frequency region is less legible because of a clutter signal with high brightness at frequencies close to 0 Hz, for example, will be described.

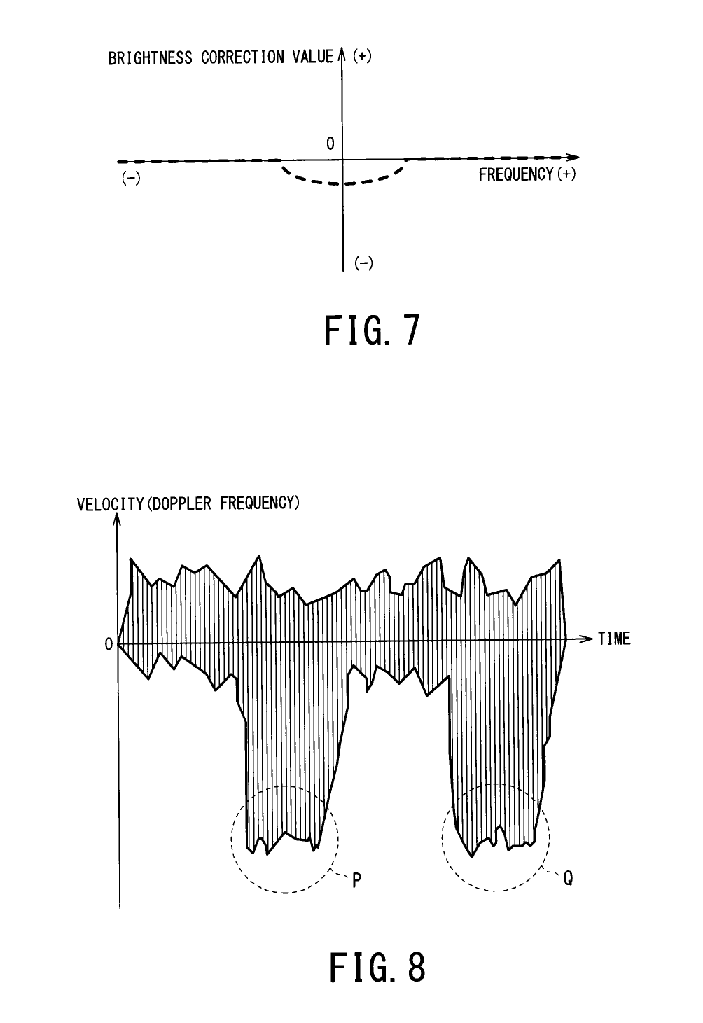

FIG. 7 is a diagram showing a first example of the method of controlling the brightness information on the waveform of the Doppler spectrum.

FIG. 7 shows an example of the brightness correction value that decreases the brightness of a signal at frequencies close to 0 Hz.

As shown in FIG. 7, in a brightness correction value line formed by a plurality of brightness correction values in the frequency axis direction, brightness correction values appropriate for frequencies close to 0 Hz are shifted by a predetermined amount in the negative direction. The system controlling unit 2a controls the brightness information on the Doppler spectrum image (Doppler spectrum) generated by the image generating unit 7 in the frequency axis direction based on the brightness correction values shown in FIG. 7.

FIG. 8 is a diagram showing a Doppler spectrum waveform in a case where a regurgitation jet signal is captured in the Doppler mode, tip end parts of the regurgitation jet signal are truncated, and the maximum flow velocity value is hard to see.

In the case where the regurgitation jet signal is captured in the Doppler mode, as shown in regions P and Q in FIG. 8, tip end parts of the regurgitation jet signal can be truncated, and the maximum flow velocity value can be hard to see. There are various possible causes of such a phenomenon. Among others, there are causes that depend on the performance (such as the S/N ratio) of the ultrasonic diagnosis apparatus itself. Although the maximum flow velocity value is hard to see with the gain value normally set in the ultrasonic diagnosis apparatus 1, the maximum flow velocity value can be displayed if the gain value or the like is set to be higher than the normal value. However, according to prior art, if the operator operates a knob for the gain value to change the setting of the ultrasonic diagnosis apparatus 1, the gain value uniformly increases over the entire frequency band. Thus, the brightness of the clutter signal with high brightness, which is the unwanted permanent echo signal described above, also increases, and the waveform of the Doppler spectrum becomes less legible as a whole. In view of this, with the ultrasonic diagnosis apparatus 1 according to the present embodiment, only the brightness correction values appropriate for high frequencies can be shifted to the positive direction.

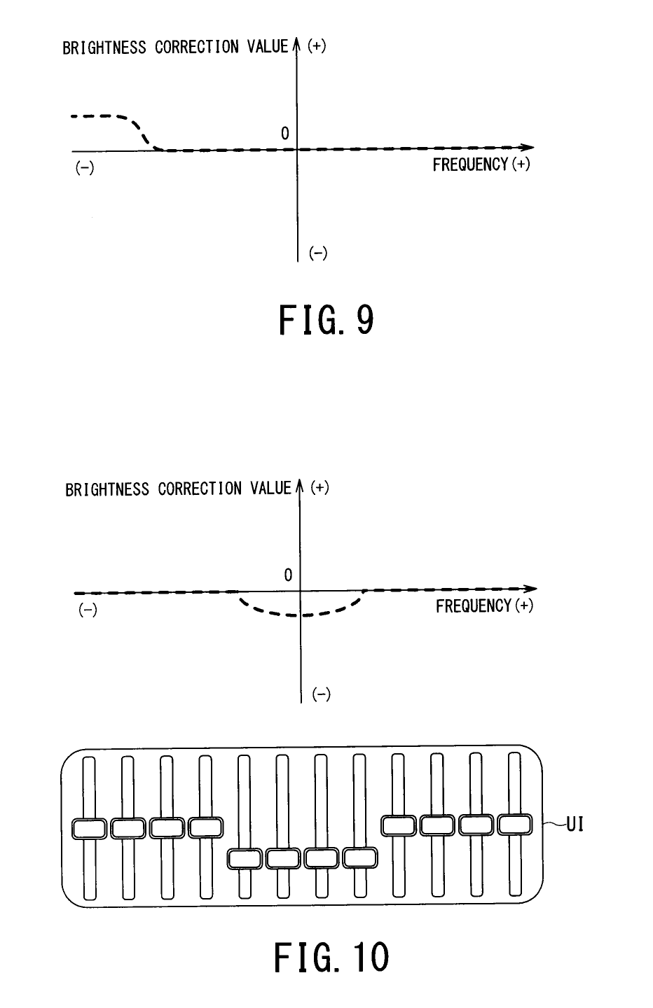

FIG. 9 is a diagram showing a second example of the method of controlling the brightness information on the waveform of the Doppler spectrum.

FIG. 9 shows an example of the brightness correction value that increases the brightness of a signal only at a high frequency region (only a high frequency region in the negative region in FIG. 9).

As shown in FIG. 9, in a brightness correction value line formed by a plurality of brightness correction values in the frequency axis direction, brightness correction values appropriate for high frequencies are shifted by a predetermined amount in the positive direction in order to increase the brightness of the signals in the high frequency region. The system controlling unit 2a controls the brightness information on the Doppler spectrum image (Doppler spectrum) generated by the image generating unit 7 in the frequency axis direction based on the brightness correction values shown in FIG. 9. In this way, in the case where the regurgitation jet signal is captured in the Doppler mode, truncation of tip end parts of the regurgitation jet signal can be prevented without increasing the brightness of the clutter signal with high brightness, which is an unwanted permanent echo signal, and the maximum flow velocity value can be made legible.

Next, a specific example of a method of operating a brightness correction value in the frequency axis direction will be described.

FIG. 10 is a diagram for illustrating a method of operating brightness correction values in the case of the controlling method shown in FIG. 7.

FIG. 10 shows a slider bar UI displayed on the display, that is, a user interface in which the entire frequency band is divided into a plurality of, such as 12, frequency domains, and the brightness correction value can be operated for each frequency domain. Of course, the number of frequency domains is not limited to 12.

In the case shown in FIG. 10, using the slider bar UI, the brightness correction value line shown in FIG. 7 is formed by shifting the brightness correction values appropriate for frequency domains close to 0 Hz in the negative direction to decrease the brightness of signals on the positive and negative sides of 0 Hz. More specifically, in order to decrease the brightness of the signals in two positive frequency domains close to 0 Hz by a predetermined amount, sliders corresponding to those frequency domains are slid. At the same time, in order to decrease the brightness of the signals in two negative frequency domains close to 0 Hz by a predetermined amount, sliders corresponding to those frequency domains are slid. The brightness correction value line formed by the sliding operation of the slider bar UI is preferably a smoothed curve as shown in FIG. 10.

Although the slider bar UI is preferably used because the slider bar allows continuous or stepwise control of the brightness information, the present invention is not limited to the slider bar, and a ON/OFF switch may be used, for example. In that case, the switch is turned on to increase or decrease the brightness by a predetermined amount.

FIG. 11 is a diagram showing an example of the user interface for operating brightness correction values in the frequency axis direction.

FIG. 11 shows a slider bar UI displayed on the display, that is, a user interface in which the entire frequency band is divided into a plurality of, such as 8, frequency domains, and the brightness correction value can be operated for each frequency domain. The operator performs a sliding operation of each slider of the slider bar UI displayed on the display from the press position shown in FIG. 11. If the touch panel 10a detects a sliding operation from the press position, the brightness information controlling unit 2a of the system controlling unit 2 controls the brightness information on the Doppler spectrum based on the brightness correction value for the involved frequency domain.

In the example shown in FIG. 11, using the slider bar UI, the brightness correction values can be changed from 0 as an initial (default) value of the brightness correction values for all the frequency domains. However, the present invention is not limited to such a configuration. The brightness information controlling unit 2a can set different initial values of the brightness correction value for different diagnosis parts. If the diagnosis part is a carotid artery, no (little) clutter component occurs, so that the initial value of the brightness correction value is set at 0 for all the frequency domains. On the other hand, if the diagnosis part is a cardiac artery, a clutter component occurs at frequencies domain close to 0 Hz, so that a negative initial value is set for the brightness correction values appropriate for frequency domains close to 0 Hz. The operator then changes the brightness correction values from the respective initial values.

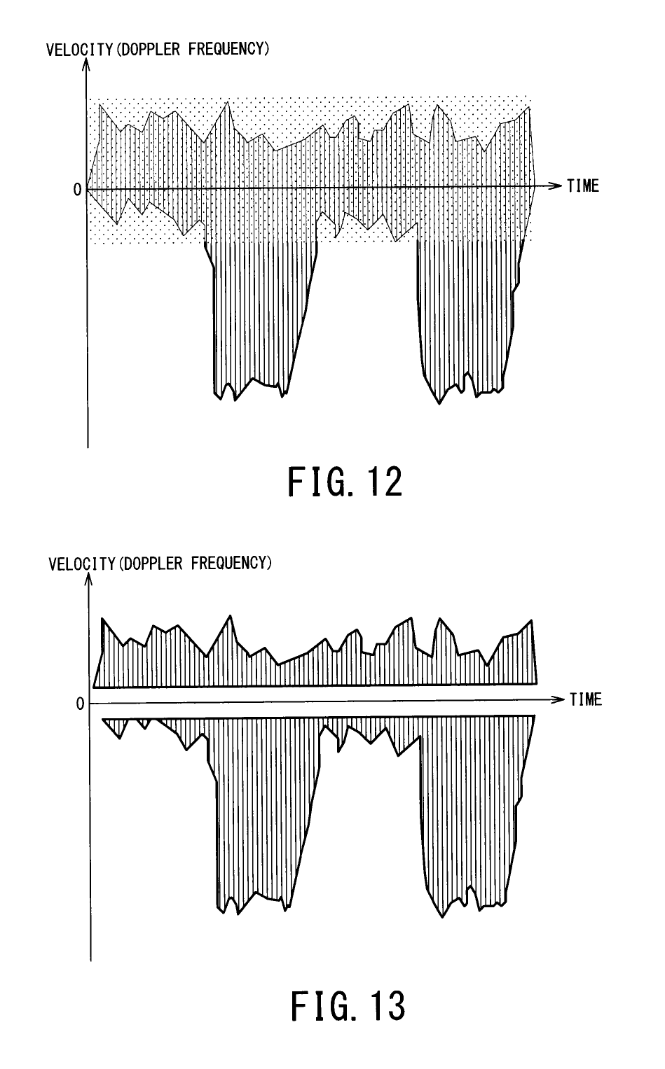

FIG. 12 is a diagram showing a waveform of a Doppler spectrum having been subjected to the brightness information control in the frequency axis direction based on the brightness correction values shown in FIG. 10. FIG. 13 is a diagram showing a case where the brightness of signals in low frequency component regions close to 0 Hz is decreased according to a conventional method that involves raising the cut-off setting of the wall filter.

In the case shown in FIG. 12, as described above, the system controlling unit 2a controls the brightness information on the Doppler spectrum image generated by the image generating unit 7 in the frequency axis direction in accordance with an instruction input by the operator via the touch panel 10a, thereby decreasing the brightness of the signals in low frequency component regions close to 0 Hz by a predetermined amount.

As shown in FIG. 13, a conventional ultrasonic diagnosis apparatus raises the cut-off setting of the wall filter in order to decrease the brightness of signals in low frequency component regions close to 0 Hz. However, depending on the setting of the wall filter, not only clutter signals but also Doppler signals (blood flow signals) from blood cells can disappear, and the time-interval measurement based on the Doppler signals cannot be accurately executed.

However, the ultrasonic diagnosis apparatus 1 according to the present embodiment can appropriately decrease the brightness of the signals in the low frequency component regions while avoiding removing the Doppler signals (blood flow signals) from blood cells required for the time-interval measurement and therefore can avoid hindering the time-period measurement based on the Doppler signals. In this case, unlike the method that involves raising the cut-off setting of the wall filter, the signals (such as the clutter signals) in the low frequency component regions are not completely removed. As a result, the operator can at least recognize the signals in the low frequency component regions and can recognize a movement of a cardiac wall or the like.

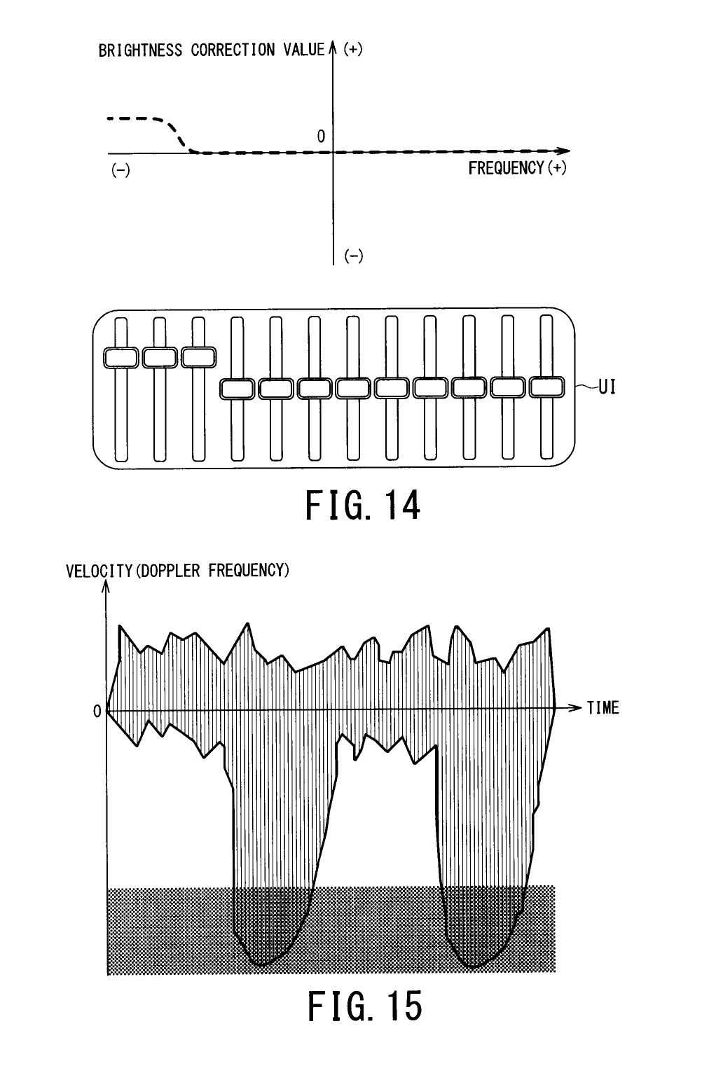

FIG. 14 is a diagram for illustrating a method of operating brightness correction values in the case of the controlling method shown in FIG. 9.

As with FIG. 10, FIG. 14 shows a slider bar UI displayed on the display, that is, a user interface in which the entire frequency band is divided into 12 frequency domains, and the brightness correction value can be operated for each frequency domain.

In the case shown in FIG. 14, using the slider bar UI, the brightness correction value line shown in FIG. 9 is formed by shifting the brightness correction values appropriate for high frequency domains in the positive direction to increase the brightness of signals in the high frequency domain. More specifically, in order to increase the brightness of the signals in three high frequency domains by a predetermined amount, sliders corresponding to those frequency domains are slid. The brightness correction value line formed by the sliding operation of the slider bar UI is preferably a smoothed curve as shown in FIG. 14.

FIG. 15 is a diagram showing a waveform of a Doppler spectrum having been subjected to the brightness information control in the frequency axis direction based on the brightness correction values shown in FIG. 14.

As shown in FIG. 15, tip end parts of the regurgitation jet signal can be prevented from being truncated and displayed in such a manner that the operator can measure the tip end parts of the regurgitation jet signal, and the operator can easily measure the maximum flow velocity value of the regurgitation jet signal.

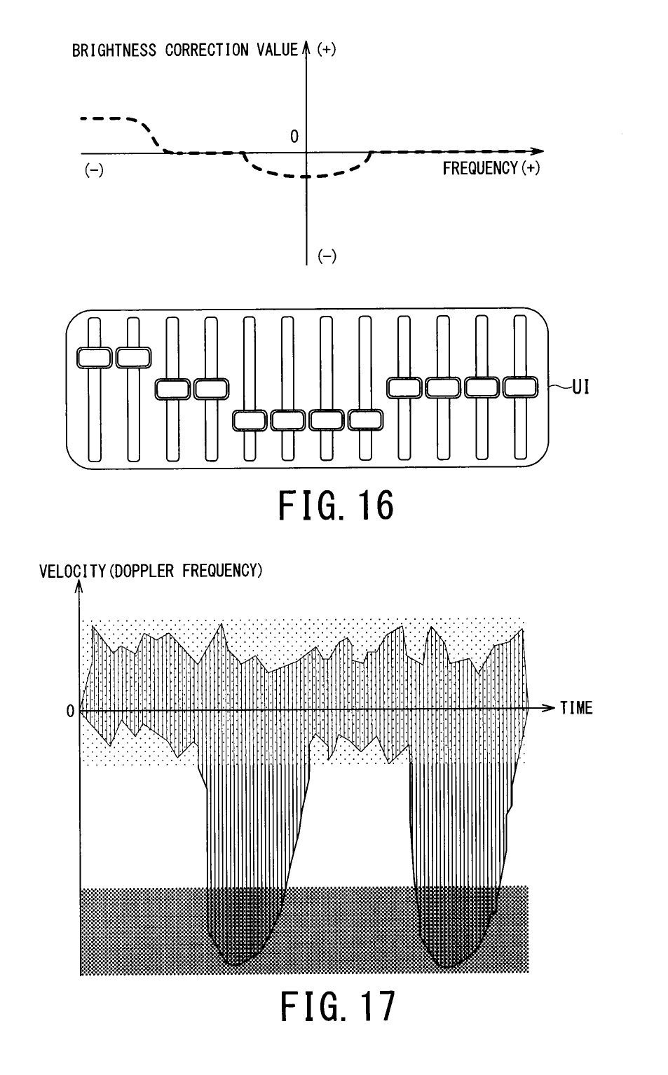

FIG. 16 is a diagram for illustrating a method of operating brightness correction values in the case of the controlling methods shown in FIGS. 7 and 9.

In FIG. 16, using the slider bar UI, a brightness correction value line that is a combination of the brightness correction value lines shown in FIGS. 7 and 9 is formed by shifting the brightness correction values appropriate for the frequency domains close to 0 Hz in the negative direction and shifting the brightness correction values appropriate for the high frequency domains in the positive direction. That is, FIG. 16 shows a case where the case shown in FIG. 7 (the case shown in FIG. 10) and the case shown in FIG. 9 (the case shown in FIG. 14) are combined with each other. The brightness correction value line formed by the sliding operation of the slider bar UI is preferably a smoothed curve as shown in FIG. 16.

Thus, the ultrasonic diagnosis apparatus 1 according to the present embodiment can appropriately decrease the brightness of the signals in the low frequency component regions while avoiding removing the Doppler signals (blood flow signals) from blood cells required for the time-interval measurement and therefore can avoid hindering the time-interval measurement based on the Doppler signals. At the same time, the ultrasonic diagnosis apparatus 1 according to the present embodiment can prevent tip end parts of the regurgitation jet signal from being truncated and make the maximum flow velocity value more legible. This is an advantage that cannot be achieved by simply uniformly raising the gain value or the like according to prior art.

FIG. 17 is a diagram showing a waveform of a Doppler spectrum having been subjected to the brightness information control in the frequency axis direction based on the brightness correction values shown in FIG. 16.

In FIG. 17, as in the case shown in FIG. 12, the brightness of the signals in the low frequency domains close to 0 Hz is decreased by a predetermined amount, and tip end parts of the regurgitation jet signal is displayed in such a manner that the operator can measure the tip end parts of the regurgitation jet signal.

Furthermore, a specific method for the system controlling unit 2a to control the brightness information on the Doppler spectrum in the frequency axis direction will be described. The brightness control (using a brightness correction value) may be a gain control that involves operating the gain value, a gamma curve control that involves operating the gamma curve value, a dynamic range control that involves operating the dynamic range value, or a map control that involves applying a color filter (a color filter of a color of pink, blue or other colors). These controls can be used singly or in combination as required.

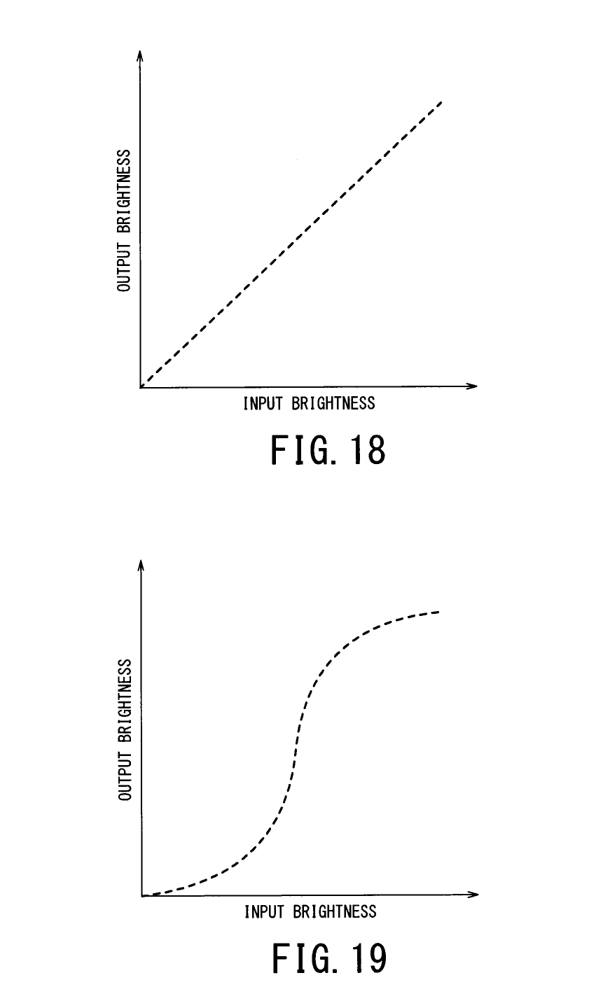

FIGS. 18 and 19 are diagrams showing specific examples of the gamma curve control that involves operating the gamma curve value.

The gamma curve control is executed in such a manner that the relationship between the input brightness and the output brightness is expressed by a straight line shown in FIG. 18 or a curve shown in FIG. 19. Thus, the brightness of the waveform of the Doppler spectrum can be corrected by executing the gamma curve control using a gamma curve circuit.

Alternatively, the brightness of the waveform of the Doppler spectrum can also be corrected by executing the dynamic range control that involves operating the dynamic range value.

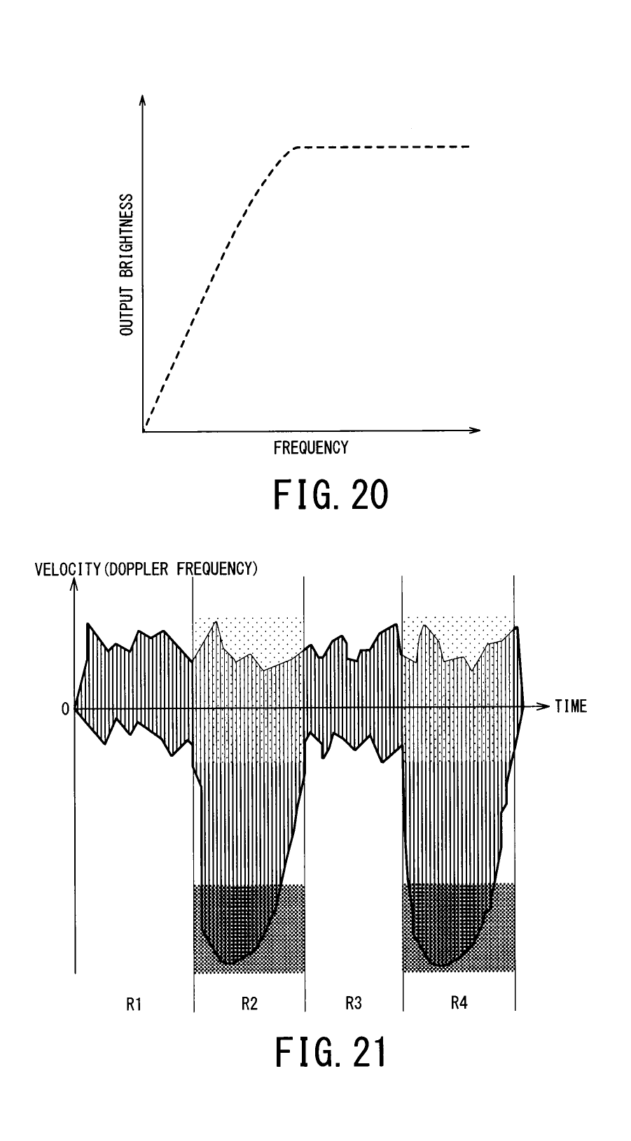

The brightness controlling method can also be achieved by executing the wall filter control. FIG. 20 is a diagram for illustrating a specific example of the wall filter control.

Referring back to FIG. 4, in step S7, the system controlling unit 2 controls the time-series data measuring unit 8 to execute various kinds of measurements concerning the Doppler spectrum (such as the time-interval measurement, the measurement of the maximum flow velocity value, the measurement of the blood volume) using the waveform of the Doppler spectrum having been subjected to the brightness control in the frequency axis direction. Since the waveform of the Doppler spectrum having been subjected to the brightness control in the frequency axis direction is used, the time-interval measurement, the measurement of the maximum flow velocity value, the measurement of the blood volume or other measurements can be accurately executed for the regurgitation jet signal. The display data generating unit 9 then merges (integrates) the measurement result from the time-series data measuring unit 8 and the Doppler spectrum image having been subjected to the bright information control under the control of the system controlling unit 2, and the display device 10 displays the resulting information.

As described above, the ultrasonic diagnosis apparatus 1 according to the present embodiment can arbitrarily control the brightness of the waveform of the Doppler spectrum and can appropriately ensure the brightness of the required signals from blood cells in the frequency axis direction while reducing the brightness of clutter signals or other unwanted permanent echo signals in a simple manner without using the wall filter or the like. Therefore, the ultrasonic diagnosis apparatus 1 according to the present embodiment can provide a waveform of a Doppler spectrum that has an envelope that can be easily traced and is easy for the operator to measure. As a result, the ultrasonic diagnosis apparatus 1 according to the present embodiment can more quickly execute measurement or other processings and improve the throughput of examination.

The slider bar UI shown in FIG. 11 can be provided not only in the frequency axis direction of the Doppler spectrum but also in the time axis direction.

FIG. 21 is a diagram showing a waveform of a Doppler spectrum having been subjected to the brightness information control in the frequency axis direction and the time axis direction.

The brightness correction values in the frequency axis direction can be made valid (ON) only in time regions R2 and R4 of time regions R1 to R4 shown in FIG. 21 in order that the brightness information can be corrected only in the time regions R2 and R4 in the same manner as the brightness information control in the frequency axis direction described above. Alternatively, the brightness control (brightness correction values) in the frequency axis direction and the equivalent brightness control in the time axis direction can also be combined (the brightness correction values in the frequency axis direction and the brightness correction values in the time axis direction can be multiplied). In this way, a waveform of a Doppler spectrum that is easy for the operator to measure can be displayed.

FIG. 22 is a diagram showing an example of the user interface for operating the brightness correction values in the frequency axis direction and the time axis direction.

FIG. 22 shows a slider bar UI displayed on the display, that is, a user interface in which the entire frequency band is divided into a plurality of, such as 8, frequency domains, and the brightness correction value can be operated for each frequency domain. In addition, FIG. 22 shows a slider bar UI′ displayed on the display, that is, a user interface in which the entire time period is divided into a plurality of, such as 15, time domains, and the brightness correction value can be operated for each time domain. The operator performs a sliding operation of each slider of the slider bars UI and UI′ displayed on the display from the press positions shown in FIG. 22. If the touch panel 10a detects a sliding operation from the press position, the brightness information controlling unit 2a of the system controlling unit 2 controls the brightness information on the Doppler spectrum based on the brightness correction value for the involved frequency domain.

As described above, the ultrasonic diagnosis apparatus 1 according to the present embodiment can arbitrarily control the brightness of the waveform of the Doppler spectrum and can appropriately ensure the brightness of the required signals from blood cells in the frequency axis direction and the time axis direction while reducing the brightness of clutter signals or other unwanted permanent echo signals in a simple manner without using the wall filter or the like. Therefore, the ultrasonic diagnosis apparatus 1 according to the present embodiment can provide a waveform of a Doppler spectrum that has an envelope that can be easily traced and is easy for the operator to measure. As a result, the ultrasonic diagnosis apparatus 1 according to the present embodiment can more quickly execute measurement or other processings and improve the throughput of examination.

The arrangement that achieves the effects described above is not exclusively provided in the ultrasonic diagnosis apparatus. Next, a case where the arrangement that achieves the effects described above is provided in an image processing apparatus (a workstation) will be described.

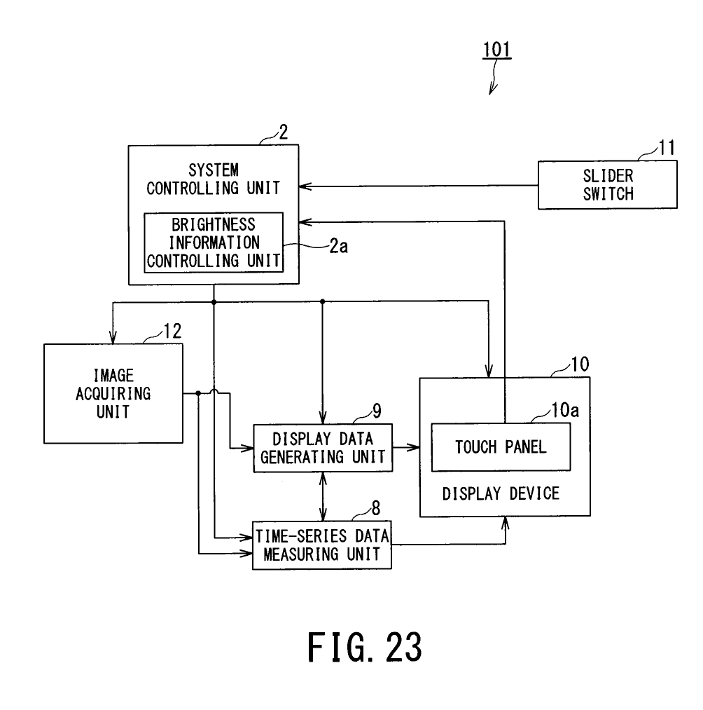

FIG. 23 is a block diagram showing a configuration of the whole of an image processing apparatus according to the present embodiment.

FIG. 23 shows an image processing apparatus 101 according to the present embodiment. The image processing apparatus 101 includes the system controlling unit 2, the time-series data measuring unit 8, the display data generating unit 9, the display device 10 and an image acquiring unit 12. The image processing apparatus 101 may be provided with the slider switch 11 for brightness information control as an alternative to or in addition to the touch panel 10a. In FIG. 23, the components of the image processing apparatus 101 that are the same as those of the ultrasonic diagnosis system 1 shown in FIG. 1 are denotes by the same reference numerals and will not be further described.

The image acquiring unit 12 acquires an ultrasonic image from an apparatus (not shown) that stores an ultrasonic image (a B-mode image, a color Doppler image, a Doppler spectrum image or an M-mode image), such as a conventional ultrasonic diagnosis apparatus or an image server. For example, the image acquiring unit 12 receives an ultrasonic image over a network, such as a local area network (LAN) of a hospital infrastructure. The ultrasonic image acquired by the image acquiring unit 12 is output to the display data generating unit 9 and the time-series data measuring unit 8 or a storage device (not shown) under the control of the system controlling unit 2.

As described above, the image processing apparatus 101 according to the present embodiment can arbitrarily control the brightness of the waveform of the Doppler spectrum and can appropriately ensure the brightness of the required signals from blood cells in the frequency axis direction and the time axis direction while reducing the brightness of clutter signals or other unwanted permanent echo signals in a simple manner without using the wall filter or the like. Therefore, the image processing apparatus 101 according to the present embodiment can provide a waveform of a Doppler spectrum that has an envelope that can be easily traced and is easy for the operator to measure. As a result, the image processing apparatus 101 according to the present embodiment can more quickly execute measurement or other processings and improve the throughput of examination.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An ultrasonic diagnosis apparatus, comprising:
a transmitting and receiving unit configured to transmit an ultrasonic wave to and receive an ultrasonic wave from a diagnosis part including a moving body in an object;
an extracting unit configured to extract a Doppler signal based on a reception signal obtained by the transmitting and receiving unit;
a generating unit configured to execute a frequency analysis on the basis of the Doppler signal and generate a Doppler spectrum;
a brightness information controlling unit configured to control brightness information on the Doppler spectrum on the basis of a brightness correction value appropriate for a frequency subjected to the frequency analysis; and
a display unit configured to display, on a display device, the Doppler spectrum on which the brightness information is controlled by the brightness information controlling unit.

2. The ultrasonic diagnosis apparatus according to claim 1, wherein
the brightness information controlling unit determines the brightness correction value for each of a plurality of frequency domains resulting from division of a frequency band.

3. The ultrasonic diagnosis apparatus according to claim 1, wherein
the brightness information controlling unit determines an initial value of the brightness correction value for each diagnosis part.

4. The ultrasonic diagnosis apparatus according to claim 1, further comprising:
an interface configured to change the brightness correction value.

5. The ultrasonic diagnosis apparatus according to claim 1, wherein
the brightness information controlling unit determines a second brightness correction value appropriate for time in addition to the brightness correction value appropriate for the frequency and controls the brightness information on the Doppler spectrum based on the second brightness correction value.

6. The ultrasonic diagnosis apparatus according to claim 5, wherein
the brightness information controlling unit determines the second brightness correction value for each of a plurality of time domains resulting from division of a time period.

7. The ultrasonic diagnosis apparatus according to claim 4, wherein
a touch panel provided on a display screen of the display device, and configured to be the user interface.

8. The ultrasonic diagnosis apparatus according to claim 4, further comprising:
a slider switch configured to be the user interface.

9. The ultrasonic diagnosis apparatus according to claim 1, wherein
the brightness information controlling unit controls the brightness information on the Doppler spectrum using at least any one of a gain control, a gamma curve control, a dynamic range control and a map control.

10. The ultrasonic diagnosis apparatus according to claim 1, further comprising:
a measuring unit configured to execute a measurement concerning the Doppler spectrum using the Doppler spectrum the brightness information on which is controlled by the brightness information controlling unit,
wherein the display unit displays the Doppler spectrum on which the brightness information is controlled by the brightness information controlling unit and a result of the measurement executed by the measuring unit.

11. An image processing apparatus, comprising:
a brightness information controlling unit configured to control brightness information on a Doppler spectrum on the basis of a brightness correction value appropriate for a frequency subjected to a frequency analysis based on a Doppler signal; and
a display unit configured to display, on a display device, the Doppler spectrum on which the brightness information is controlled by the brightness information controlling unit.
